(19) United States
(12) Patent Application Publication
(10) Pub. No.: US 2010/0234834 A1
(43) Pub. Date: Sep. 16, 2010

(54) Device for localized thermal ablation of biological tissues, particularly tumoral tissues or the like

(75) Inventors: Marco Mattiuzzi, Grassina (Bagno a Ripoli), Firenze (IT); Gaetano Cascini, Firenze (IT); Davide Russo, Firenze (IT)

Correspondence Address: Themis Law, 7660 Fay Ave Ste H-535, La Jolla, CA, 92037, US

(73) Assignee: BRACCO IMAGING S.P.A., Milano (IT)

(21) Appl. No.: 12/305,972

(22) PCT Filed: Jun. 28, 2007
(86) PCT No.: PCT/EP07/56506

§ 371 Date: Feb. 22, 2009

(30) Foreign Application Priority Data
Jul. 4, 2006 (EP) 06425461.8

(51) Int. Cl. A61B 18/22

(52) U.S. Cl. 606/15

(57) ABSTRACT

Device for localized thermal ablation of lesion tissues, particularly tumoral tissues or the like, which device comprises: a probe or needle intended to be positioned with the end tip at the lesion tissue or tumoral tissue area to be removed; which probe or needle support at least a light guide as an elongated member like a thin wire or thread, one of the ends thereof is an end emitting heating electromagnetic energy and which light guide ends at said end of the probe or needle by a tip irradiating said electromagnetic energy, particularly as a laser light and the other end thereof is connected to a source generating the electromagnetic energy; means for controlling the activation/deactivation of the source generating the electromagnetic energy. Characterized in that in combination it comprises means for the controlled distribution of the heating action on the lesion tissue area generated by the electromagnetic energy emitted by the irradiating tip inside a volume having a predetermined size. The invention relates also to a method for localized thermal ablation.

The present invention relates to a device for localized thermal ablation of lesion tissues, particularly tumoral tissues or the like, which device comprises:

a probe or needle intended to be positioned with the end tip at the lesion tissue or tumoral tissue area to be removed;

which probe or needle support at least a light guide as an elongated member like a thin wire or thread, one of the ends thereof is an end emitting heating electromagnetic energy and which light guide ends at said end of the probe or needle by a tip irradiating said electromagnetic energy, particularly as a laser light and the other end thereof is connected to a source generating the electromagnetic energy;

means for controlling the activation/deactivation of the source generating the electromagnetic energy.

These type of devices are known and are used in medical field particularly for ablating tumoral tissues. The great advantage of these devices is that the ablating action is little invasive and periods in bed of the patient are very short.

At present various studies have been made showing a very good result of these devices for removing small tumoral masses with a diameter of about 10 mm. Instead as regards greater tumoral masses, the treatment thereof is still difficult and however the complete removal of the tumoral tissue requires the movement of the probe and other expedients.

The light guide is generally composed of a filament of optical fiber with the great advantage of having a reduced attenuation of conveyed luminous energy and allowing to restrict the invasive effect of the needle or probe by the fact that the needle or probe wherein the end portion of the light guide is inserted can have very small diameters. On the other hand that is advantageous considering the fact that some tumoral lesions may be in anatomical districts crowded with organs having functions also of the vital type so the insertion of the probe or needle is related to relatively high damage elements.

The light source is generally composed of a laser light source suitable to provide the necessary intensity to increase temperature of irradiated tissue up to levels necessary for the treatment.

However the practical use of thermal ablation by devices irradiating electromagnetic energy is still subjected to further problems especially occurring when treating very large lesion tissue areas. In this case, the problem of treating a large lesion can be considered to be solved simply by a marking of the tip of the probe or needle for example by various imaging means like ultrasound ones or the like and so by moving the needle or probe with respect to the tissue area to be treated to such an extent necessary to treat said tissue area along its whole size. However in this case there is the problem of moving the needle or probe with respect to the area to be treated and so by orienting the tip irradiating the electromagnetic energy in order to irradiate a different portion of said area only when the area currently subjected to irradiation has been completely treated. Therefore there is the problem of determining when a partial area belonging to a larger area of lesion tissue has been treated in a way sufficient to determine desired therapeutic effects. In addition to this problem there is also a further problem that has been found in such thermal ablation devices operating with irradiation of electromagnetic energy and it is the fact that water vapour is generated due to tissue heating. The presence of vapour makes impossible to use a simple and inexpensive technique such as ultrasound images for monitoring the treatment. Ultrasounds are just blinded with the presence of vapour.

Document US-B1-6 302 878 discloses a method and a device for termoablation of tumoral tissues in which there are provided means for deviating the optical flux. In this case reflecting means are used which are able to focus or deviate the emitted electromagnetic energy.

Document EP-A-0598984 Discloses different measures for deviating the optical flux in different directions and for different medial treatments in order to reach regions which are difficult to be reached. This deflection means are formed by several slots having reflecting properties are by a certain amount of particles having reflecting properties and which particles are distributed in a spherical containment volume.

Document US-A-5 454 807 discloses a system and a method for termal treatment of the lesion tissues in which a cannula is used for carrying out the treatment. The cannula is arranged for the passage of a cooling fluid also in this document different arrangements are disclosed for deviating the electromagnetic beam. As a particular solution mirrors are used which can have different shapes depending on the particular case to be treated, so that the electromagnetic beams and the cooling fluid are deviated in order to operate on different surfaces of the lesions.

Document EP-A-0411132 discloses a device for the termal treatment of lesion tissues which is provided with a inflatable ball at the tip of the cannula or of the needle. The ball is filled with a liquid in which a certain amount of reflecting particles are solved.

None of the said systems provides for a way to concentrate the electromagnetic energy and thus the heating action on a particular portion of the region to be treated and depending on the temperature of the said portion to concentrate the electromagnetic energy on a different portion.

From a more general point of view, as regards thermal ablation by irradiating with laser light the lesion tissue there is the problem of obtaining an even distribution of the heating effect on a large area or anyway on an area or volume that are larger than the tissue area directly exposed to the output end of the guide fiber of the light ray, so called fiber tip or tip. The area directly adjacent to the output end of the guide fiber of the laser beam is a small area or very small volume so the heating action is very deep in this small area whereas the heating action quickly decreases as the distance from said output end increases. Therefore there is the risk of exerting an excessive heating action in the areas directly adjacent to the tip, and of treating more distant areas in an insufficient way. Therefore it is necessary to have a means for learning or controlling the real spatial distribution of the heat or heating effect in the volume surrounding the tip and depending on the distance from the tip in order to control the heating effect and therefore the desired treatment.

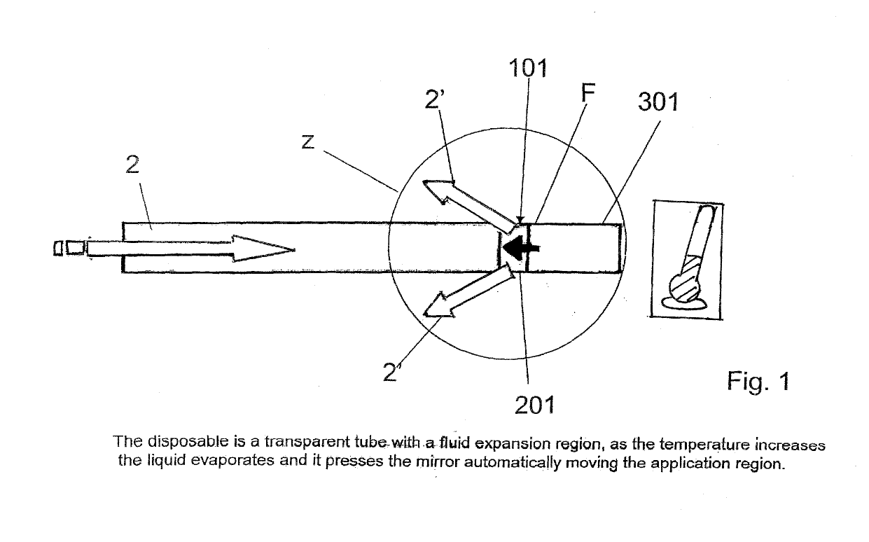

Various studies have been made in order to understand interactions between tissues and electromagnetic energy, particularly as laser beams. FIG. 1 very schematically shows how the energy of laser irradiation on a tissue in the process called laser volatilization of tissues is considered to work. It is a process used for incision and tumoral ablation. During tissue removal three steps have been pointed out which have been defined as follows even referring to the temperature range taken by the tissue: coagulation defines the tissue heating condition at temperatures between 55-100° C.: water vaporization defines the heating step between 100 and 400° C.; combustion occurs when heating exceeds 400° C. It has been found that tissue mass loss is due firstly to radiating flux of the treated area. As regards fluxes with values exceeding 1000 J/cm², the pointed out effect corresponds to a whitening of the tissue. The phenomenon so called of popcorn vaporization occurs at thermal energy fluxes between 1100 and 1500 J/cm², while the carbonization and combustion occurs when the radiating flux exceeds 1500 J/cm².

Very in-depth studies have been made in order to determine how the heat is distributed in tissues and particularly in interfaces between healthy tissues and lesion tissues. Essentially studies have found that parameters determining the distribution of heat are complex and that it is not possible to generalize or isolate a general law that can be adapted to all conditions even approximately. Particularly the way according to which thermal diffusion works in tissues after irradiation by laser light depends not only on the radiating flux but also on the tissue quality referring above all to absorption of the electromagnetic irradiation by tissues. In this case, each different type or kind of tissue has a different behaviour and so it is difficult to determine a priori a general law.

As regards vapour production at the moment the problem is not dealt with except by trying to modulate the energy supply, in order to have the greatest heating effect on tissue without generating vapour, by using the different response to irradiation of different tissue components, water among thereof. A solution adopted to avoid vapour formation is the alternative operation of laser source and so irradiating tissues by laser light pulses. However the solution is not a satisfactory one since the thermal ablation effect however is small with respect to what could be obtained by a constant and adjusted irradiation.

Therefore the invention aims at improving known thermal ablation devices of the type described hereinbefore allowing firstly to overcome drawbacks of known methods and that is the fact of allowing in a substantially simple and safe way to treat relatively large lesion tissue areas without the risk of burning some partial areas or of heating in an insufficient way other partial areas and at the same time allowing to treat the whole size of the lesion tissue as safely as possible.

A further aim is the fact of allowing what has been mentioned above by automatic or nearly automatic means reducing as much as possible a direct controlling intervention by the operator and allowing to standardize the ablation process.

The invention achieves the above aims by providing a device of the type described hereinbefore wherein in combination means for controlled distribution of the heating action on the lesion tissue area generated by the electromagnetic energy emitted by the irradiating tip inside a volume having a predetermined size are comprised.

The said means consisting in means for varying the distribution of the emitted electromagnetic energy on at least part of the lesion tissue area as a function of the temperature measured at one or more places of the lesion tissue area and particularly at the lesion tissue area currently under treatment.

The said means for the controlled distribution of the heating action on the lesion tissue area consist in means for distributing, projecting or pointing the electromagnetic irradiation emitted by the irradiating tip on different portions of the lesion tissue area, the said means for distributing, projecting or pointing the electromagnetic irradiation emitted by the irradiating tip being adjustable relatively to the portion of the lesion tissue area to be irradiated as a function of the temperature of one or more portions of lesioned tissue area, particularly as a function of the temperature of the portion of lesioned tissue area currently under treatment and in such a way that by reaching of a predetermined temperature at the portion of the lesioned tissue area under treatment the said means distributing, projecting or pointing the electromagnetic irradiation emitted by the irradiating tip are automatically adjusted to distribute, project or point the electromagnetic irradiation on a different portion of the lesion tissue area.

According to a further embodiment which can be provided either separately as in combination with the above one, the means for the controlled distribution of the heating action on the lesion tissue area generated by the electromagnetic energy emitted by the irradiating tip inside a volume having a predetermined size consist in means for automatically varying the intensity and/or the spectrum of the emitted electromagnetic energy as a function of the temperature in the irradiated lesion tissue area.

As it appears as regards said means for controlled distribution of the heating action it is possible to use different solutions that can all be made according to a form reduced in size and that can be integrated in a thermal ablation probe or needle by electromagnetic irradiation particularly laser light.

The first general embodiment advantageously provides to use means for controlled distribution of the heating action generated by the electromagnetic energy on the lesion tissue area composed of active means for distributing, projecting or pointing the electromagnetic irradiation emitted by the irradiating tip on different portions of the lesion tissue area.

In this case, the ray or beam emitted by the irradiating tip, for example the laser beam emitted by the end of an optical fiber is deflected by diffusion optical means for example a transverse diffuser or by reflecting or projecting means from the direction of propagation that it has when it exits from the optical fiber or other guide. The deflection, reflecting, projection or diffusion effect may be made independent from the temperature of the means or a treated tissue area both as regards intensity, direction and impression of the beam or ray. That can be automatically obtained in various ways for example by using supporting means for reflectors, projectors, diffusers changing the shape and/or size according to temperature. By means of such mechanical distortions that generally are reversible and repeatable, such as the case of shape memory materials, it is possible to change the orientation of reflectors or other projecting or diffusing means for the laser beam.

Therefore particularly the invention provides said means for distributing, projecting or pointing the electromagnetic irradiation emitted by the irradiating tip on different portions of the lesion tissue area composed of means for diffusing or concentrating or reflecting the electromagnetic irradiation which change depending on the temperature the direction and/or impression of a ray or beam of electromagnetic irradiation and the portion of the lesion tissue area illuminated by said ray or beam or upon which said ray or beam of electromagnetic energy is incident.

Among these ones means diffusing, concentrating or reflecting the electromagnetic irradiation which are supported in an orientable way by means of supports whose shape and/or size depends on the temperature or changes according to temperature are advantageous and which supports are in thermal contact with the surrounding environment and/or are subjected to heat due to irradiation by the electromagnetic irradiation coming from the irradiating tip.

In a particular embodiment the irradiating tip comprises at least a reflecting member that is supported in an orientable way with respect to the direction of propagation of the beam coming from said irradiating tip and/or to the direction of the axis of the light guide and whose orientation is controlled by mechanical thermal sensitive means.

In this case means for controlling the orientation of the reflecting means are for example advantageously composed of a thermal sensitive shape memory member or of a bimetallic member or a member bending by a predetermined angle or a predetermined bend depending on temperature.

A variant embodiment provides the reflecting member to be supported in a way movable in the direction of propagation of the electromagnetic ray and depending on temperature being mounted on supporting means changing their length according to temperature.

When there are considered particularly means diffusing the electromagnetic irradiation for example a laser beam having a specific direction of propagation and which is diffused transversally to such direction of propagation from diffusing means, so the means diffusing the electromagnetic irradiation can have a predetermined size and can be composed of a substance having a transparency and/or a diffusion index that change depending on temperature. Said substance takes different conditions for diffusing the electromagnetic irradiation in different areas of its size according to the local temperature in said areas.

For the diffusing member it is advantageous to be composed of a member elongated in the direction of propagation of the electromagnetic beam from the irradiating tip or longitudinal axis of the guide propagating the electromagnetic irradiation, said elongated member being composed of a material intended to diffuse the electromagnetic irradiation through the peripheral shell wall and said material being provided with a diffusion index that changes depending on the temperature between a smallest and greatest diffusion value of the input electromagnetic irradiation.

As an alternative the diffusing member can be covered externally that is along its surface emitting the electromagnetic irradiation by a material layer having a transparency or an opacity that can change depending on temperature.

A particular embodiment of the device according to the present invention provides the diffusing member to be composed of a plurality of various diffusing members arranged one near the other and having different functions of the dependence between diffusion index and temperature.

Still another different embodiment of the invention can provide means for distributing the heat generated by the electromagnetic irradiation on distributing means.

Different embodiment variants are possible.

A first variant comprises means for distributing the heat generated by the electromagnetic irradiation upon them that are solid mechanical means transmitting the heat generated by the electromagnetic irradiation emitted by the irradiating tip. These means can be advantageously composed of one or more wires, bands or flaps axially projecting in the direction of propagation of the electromagnetic irradiation past the irradiating tip in order to make an umbrella and which wires, bands and/or flaps are composed of a material that can be deformed according to temperature, the irradiating tip being provided with means for orienting and/or transmitting the electromagnetic beam on said wires, bands and/or flaps and/or inside thereof such that when said wires, said bands and/or said flaps progressively get warm, they change their shape and/or move in order to be open wide one with respect to the other or they move angularly radially outward.

A further variant provides to heat a fluid present inside the area or region to be treated and provides said heated fluid to be moved in the area to be treated, thus conveying the stored heat. Particularly said fluid is composed of vapour spontaneously made when heating tissues by the electromagnetic irradiation and which is due to the presence of water in tissues.

In this case, by means of the invention, the vapour that is considered as a drawback of the thermal treatment by irradiation of electromagnetic irradiation, becomes a heat carrier in order to obtain an heating effect even and widespread on all the volume to be treated.

The movement of vapour or other fluid may occur by pushing means such as a fluid jet or fluid or vapour blow or by sucking vapour or carrying fluid.

As an alternative means for pushing said fluid can also be composed of the mechanical pressure wave generated by a source of acoustic waves particularly ultrasound ones. In this case low frequency ultrasound waves and with triangular or sawtooth pulse arrangement are advantageous.

As mentioned above a particular embodiment provides as the thermal storing fluid for transporting the thermal energy the vapour generated by heating the tissue by the electromagnetic beam coming from the irradiating tip.

In combination with said means controlling the distribution of the heating effect it is possible to provide also means for detecting physical parameters of the lesion tissue changing according to temperature of the lesion tissue, such to check the occurred treatment of portions of a very large lesion tissue area. In this case the measurement can be provided in combination with automatic means moving the needle or probe for treating a different portion and/or with the operation of one or more means already described above referring to one or more different variants.

In this case the device according to the present invention comprises at least a sensor measuring physical parameters of the lesion tissue depending on heating temperature thereof, which sensor is supported at a certain distance from the irradiating tip in a predetermined position with respect thereto and which sensor measures the change of said physical parameter of the lesion tissue comprised between said irradiating tip and the sensor and means for processing the measurement signal of the sensor which determine the heating temperature of said lesion tissue area on the basis of said measurement signal, as well as signalling means and/or possibly also automatic means for modulating the electromagnetic beam and/or automatic means for moving the irradiating tip operated on the basis of said measurement signal.

Alternatively or in combination the sensor can be of the electric, temperature, acoustic, optical, laser, chemical, electrochemical, luminescence, RF wave change, pH, position, micro-movement, selective-tissue type.

For a predetermined type of lesion tissue in a predetermined anatomical district the correlation function between heating effect, heat diffusion and change in the physical parameter to be measured is determined, said function being sampled and stored in a table comparing and evaluating signals generated by the sensor measuring said physical parameter.

According to a further additional characteristic of the invention that can be provided in combination with one or more of the preceding described embodiments, means for distributing the heat generated by the electromagnetic irradiation are provided composed of substances intended to be distributed or diffused or which permeate in time the tissue area to be treated and which substances are heated by the laser irradiation and distribute the heat by perfusing the tissue to be treated. In this case, the irradiating tip of the needle comprises a nozzle 7 for localizely injecting a substance intended to distribute the heat or to adjust or make even the heating action.

According to a first variant injection means provide to inject a heat thermoregulating/storing substance, particularly a substance having a predetermined change of state temperature for example from liquid to gaseous and/or from solid to liquid or vice-versa and which temperature corresponds to the thermal treatment temperature of the lesion. In combination with said substance means for containing and/or retaining said substance in a predetermined volume and/or outside it, particularly around a predetermined volume can be provided, which volume approximately coincides with the volume wherein the lesion tissue to be treated by thermal ablation is provided.

In this case the containing action of the thermoregulating substance can be obtained by a ferromagnetic behaviour thereof or by associating said substance to a conveying carrier composed of a substance with ferromagnetic properties. It is therefore possible also to provide means for generating a localized magnetic field having such a spatial position and size to permeate only the lesion tissue area and/or to surround the lesion tissue area to be treated, in order to distribute the thermoregulating substance in the volume corresponding approximately to the tissue area to be treated or around it along the surface enveloping said tissue area to be treated.

Referring to this last variant it is particularly advantageous when the thermoregulating/storing substance is a substance that works as a barrier of the heat propagation outside the lesion tissue area to be treated, the generated magnetic field being such that the ferromagnetic carrier concentrates the thermoregulating substance in an enveloping jacket of said lesion tissue area to be treated and the thermoregulating substance being provided with a vaporization or fusion temperature of 35 to 38° C.

The thermoregulating substance and/or the substance with ferromagnetic properties can be also contained in micro-bubbles or micro-balls and/or micro-bubbles or micro-balls can be the thermoregulating substance and/or the ferromagnetic substance.

A further variant provides that by means of injecting means a heat storing fluid is locally provided, particularly a substance having a predetermined temperature of the change of state as from liquid to gaseous and which temperature corresponds to the thermal treatment temperature of the lesion, which injector comes out at the output emitting the electromagnetic beam of the irradiating tip there being provided means for mechanically pushing said fluid.

The mechanical pushing of the heat storing fluid can be obtained in various ways, for example means for pushing said fluid can be composed of a direct conveying carrier composed of the natural lymphatic or vascular flow.

As an alternative to or in combination means pushing said fluid can be composed of a fluid jet there being provided on the tip of the probe or needle at least a nozzle supplying said jet or said jets.

Again as an alternative to or in combination means for pushing said fluid can be also composed of the mechanical pressure wave generated by a source of acoustic waves particularly ultrasound ones. In this case low frequency ultrasound waves and with triangular or sawtooth pulse arrangement are advantageous.

A particular embodiment provides as the thermal storing fluid for transporting the thermal energy the vapour generated by heating the tissue by the electromagnetic beam coming from the irradiating tip.

Again according to a possible variant embodiment as regards the substance controlling the thermal diffusion it provides the use of substances that can modify the vascular and/or lymphatic circulation in the area corresponding to the lesion tissue. In such variant means for controlling the vascular or lymphatic circulation are advantageously composed of magnetorheological substances there being provided means generating localized magnetic fields operating magnetorheological substances to make agglomerates for locally preventing vascular and/or lymphatic flow generating a barrier to thermal diffusion by perfusion. A variant can be composed of means locally coagulating the blood in the lesion tissue area.

In this case an advantageous change of functions of thermal transfer from irradiating tip of the probe or needle in the direction moving away therefrom is obtained.

It is to be noted how the different embodiment variants and the different ways of making them described above can be provided in any combination or sub-combination one with the other when they are technically compatible in order to make the control of the heating distribution on all the size of the lesion to be removed as an abounding one and a more safe one.

The invention relates also to a method for localized thermal ablation of lesion tissues, particularly tumoral tissues or the like, which method comprises the following steps:

Generating an electromagnetic irradiation having a predetermined energy and frequency;

Irradiating locally and for a predetermined period of time, with said electromagnetic irradiation, a lesion tissure area or a portion thereof in order to increase the temperature of the lesion tissue of said area or portion thereof up to a predetermined value;

Characterized in that in combination it comprises steps controlling in an active way the distribution on the lesion tissue of the heating action generated by the electromagnetic energy inside a volume having a predetermined size.

A first embodiment of said method provides that the control of the distribution of the heating action generated by the electromagnetic energy on the lesion tissue area is carried out by an automatic control for distributing, projecting or pointing the electromagnetic irradiation on different portions of the lesion tissue area depending on the temperature reached by said areas.

Particularly the control of the distribution, projection or pointing of the electromagnetic irradiation on different portions of the lesion tissue area is obtained by changing diffusion or concentration or projection or reflection parameters of the electromagnetic irradiation depending on the temperature such as the direction and/or impression of a ray or beam of electromagnetic irradiation and the portion of lesion tissue area illuminated by said ray or beam or upon which said ray or beam of electromagnetic energy is incident.

The change of the direction of the electromagnetic irradiation ray and/or of the area illuminated by diffusion, is obtained by mechanical means whose shape and/or size depends on the temperature or changes depending on the temperature and the latter thermally contact the surrounding environment and/or are subjected to heating by irradiation by the electromagnetic irradiation.

According to a variant embodiment that can be provided as an alternative or in combination, the change in the diffusion of the electromagnetic ray is obtained by a change of the transparency and/or a diffusion index that can change depending on the temperature of a diffusing member taking different diffusion conditions for the electromagnetic irradiation in different areas of its size depending on the local temperature in said areas.

A further embodiment of the method according to the invention provides the distribution of heat generated by the electromagnetic irradiation to be changed by moving heat transmitting means in the lesion tissue area.

In a first variant embodiment the movement of heat transmitting means is made by the shape memory effect depending on the temperature of heat transmitting means generated by the electromagnetic irradiation thereon.

In an alternative variant of the method the control of the distribution of heat is obtained by moving a fluid transporting the stored heat. The fluid is advantageously composed of vapour generated by thermal treatment of tissues that are object of the treatment. The movement may be carried out for example by blowing and/or sucking the vapour or by transportation by means of pressure waves, such as ultrasound pulses.

Further improvements of the device and method according to the present invention are object of subclaims.

Characteristics of the invention and advantages derived therefrom will be more clear by the following description of some not limitative embodiments schematically shown in annexed drawings, wherein:

FIG. 1 is a first version of a second embodiment of the invention wherein means for actively distributing, projecting or pointing the laser irradiation ray or beam are automatically deviated in order to treat different portions of a larger area wherein a lesion tissue is localized and which embodiment provides means for moving a mirror projecting the laser irradiation ray or beam which are composed of a fluid subjected to thermal expansion.

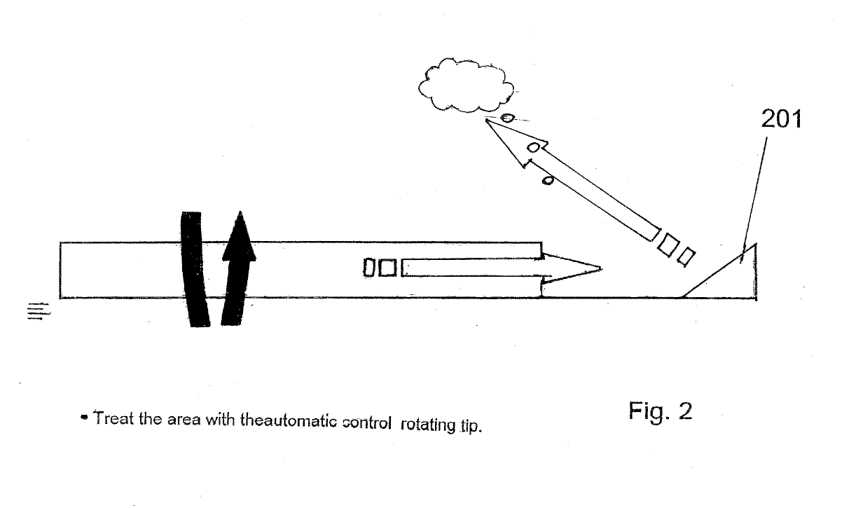

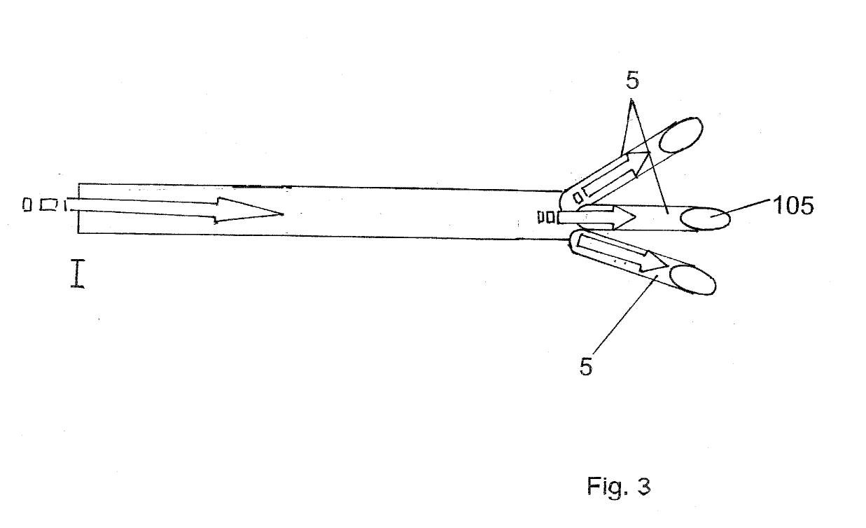

FIG. 2 similarly to FIG. 3 is a further embodiment variant of the said second embodiment.

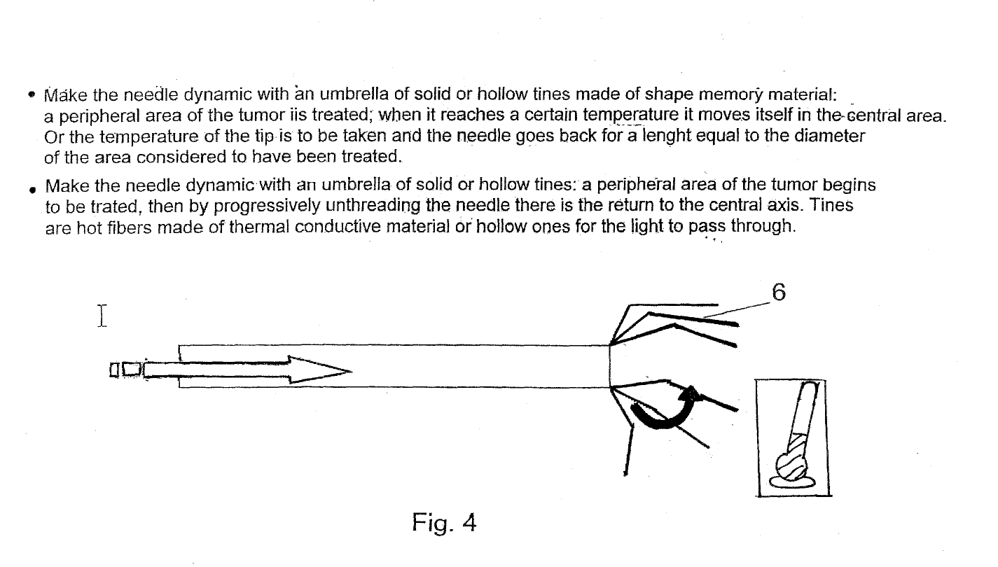

FIGS. 3 and 4 are two further variant embodiments of the device according to the second embodiment of the present invention.

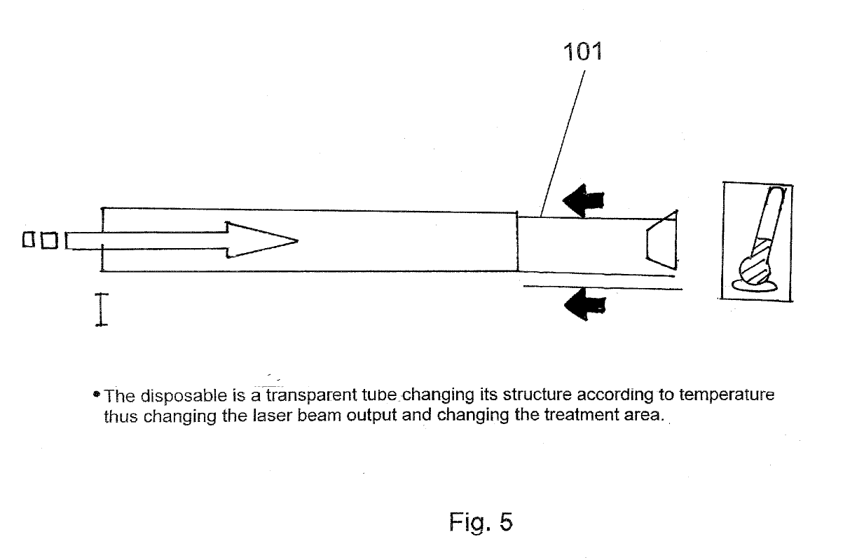

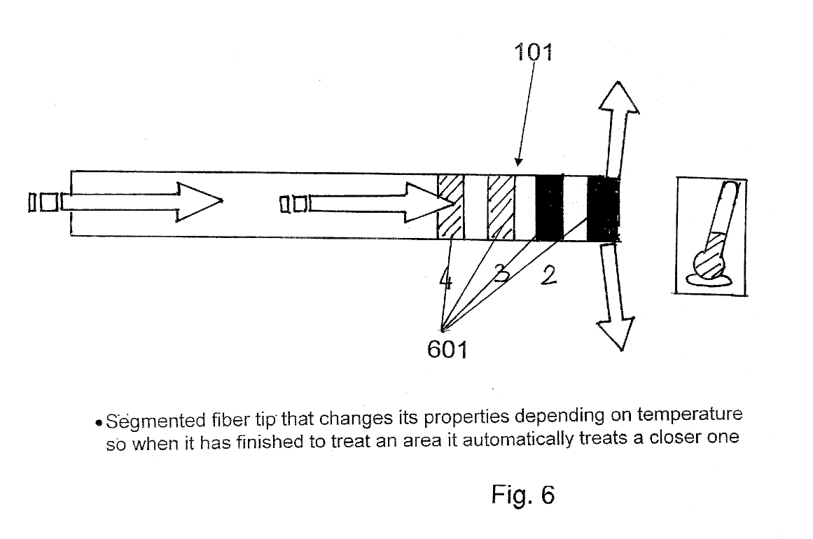

FIGS. 5 and 6 are two variants of a third embodiment of the invention wherein the temperature affects the structure of the irradiating tip changing the laser light output depending on the temperature and so the area upon which the laser beam is projected.

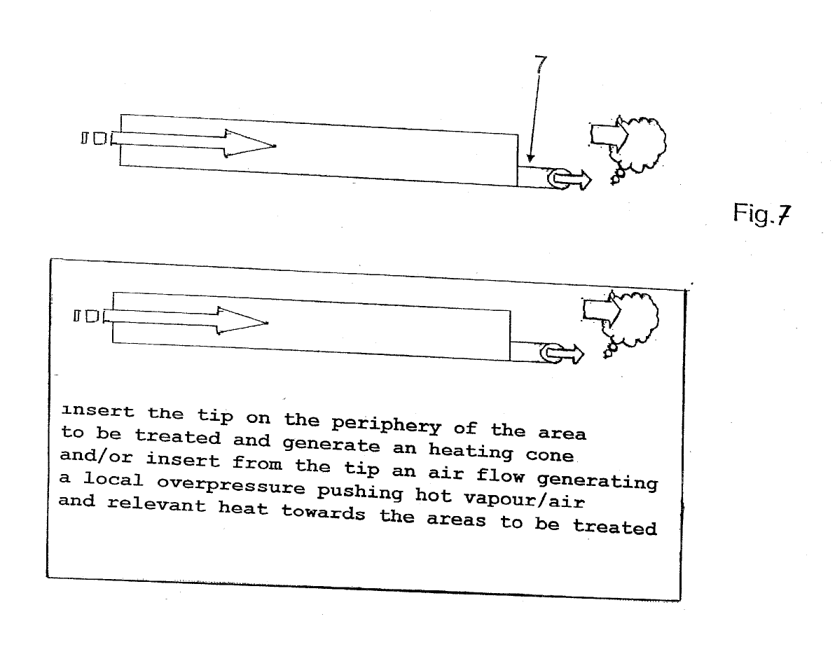

FIG. 7 is an embodiment of the invention wherein means for distributing the heat generated by the electromagnetic energy are composed of substances intended to be distributed or diffused or that permeate in time the tissue area to be treated and which substances are heated by the laser irradiation and distribute the heat by perfusing the tissue to be treated.

Referring to figures, probes or needles for thermal ablation of lesion tissues are schematically shown, particularly for tumoral tissues by heating with laser light.

The structure of these probes or needles is known per se and various figures as a principle show arrangements that the invention aims to make in order to solve above prior art problems.

Particularly current methods and devices for thermal ablation by heating with laser light are described for example in following documents: “Low Power Interstitial Photocoagulation in rat Liver, Proc. of SPIE Vol. 1882, Laser-Tissue Interaction IV, ed S. L. Jacques, A. Katzir 8 Luglio 1993) Copyright SPIE”, and U.S. Pat. No. 4,592,353, U.S. Pat. No. 4,692,244, U.S. Pat. No. 4,736,743.

Referring particularly to FIG. 1, this figure shows the end of a needle or probe for thermal ablation corresponding to the tip irradiating a laser radiation beam or ray. As it results from above mentioned documents the latter is generated by a laser source and it is transmitted through a thin optical fiber to the end of the needle or probe at which the irradiating tip is provided. In FIG. 1, the needle and the fiber are denoted together by reference number 1, while the arrow denotes the direction of transmission of the laser radiation ray or beam. The irradiating tip at which the irradiation comes out and by means of which it is directed against an area to be treated is denoted by 101.

Referring to FIG. 1, there is shown a first embodiment of a probe or needle for thermal ablation by laser irradiation having means for controlling the distribution, projection or pointing of the electromagnetic irradiation on different portions of the lesion tissue area that is obtained by a change depending on the temperature of electromagnetic irradiation distributing or concentrating or projecting or reflecting parameters such as the direction and/or impression of an electromagnetic irradiation ray or beam and the portion of the lesion tissue area illuminated by said ray or beam or upon which said electromagnetic energy ray or beam is incident.

In this embodiment the needle or probe ends by an irradiating tip 101′ comprising a reflecting mirror 201 oriented such to reflect the output laser irradiation according to one or more directions, for example along a conical beam. In this case, the mirror 201 receives the laser irradiation indicated by the arrow 2 and reflects it back as indicated by arrows 2′. The mirror 201 is supported in a sliding extension provided in the irradiating tip and indicated by 301 which extension is filled for example with a fluid. Depending on the temperature attained by the tissue surrounding it the fluid gets warm and expends pushing the mirror 201 in the direction of arrow F, therefore the reflected irradiation is progressively oriented towards different portions of the lesion tissue area Z to be treated schematically highlighted by circle Z. The shown condition is the one with the mirror reaching its extreme position corresponding to the linear movement as great as possible. Obviously the extension of the mirror travel can be determined by a limit stop that can be also possibly provided as a movable one depending on the greatest provided extension of the area to be treated.

In this case, the needle or probe are further provided with a flexible control for pulling or pushing said limit stop means allowing the movement.

As regards the way of use, by providing an embodiment such as the one shown in FIG. 1, in the initial condition when no heating action has taken place, the mirror would be moved in the limit stop position to the right of FIG. 1 that is close to the outermost end of the irradiating tip. Thermal action exerted by the irradiation first heats the areas more close to the irradiating tip wherein the fluid is contained that begins to expand pushing the mirror to the left.

By arranging the irradiating tip in the condition of initial treatment on the right border delimiting the treatment area Z that is on the end side of the irradiating tip 101, the movement of the mirror occurs in the direction progressively approaching it to the border diametrically opposite of the area z to be treated. By defining the movement limit stop in position corresponding to said border area an automatic movement of the mirror is obtained allowing to automatically distribute the laser irradiation on the whole area Z to be treated and without intervention of any persons obtaining an heating effect substantially evenly distributed on the whole volume to be treated or on a great portion thereof.

In the variant of FIG. 2, the mirror 201 is mounted on an automatic control rotating support. Particularly, in order to allow both the automatic rotation and the automatic axial movement, means supporting said mirror can be combined with extensible supporting means depending on the temperature therefore involving the axial movement of the mirror and means extensible due to the thermal heating action and controlling rotating supporting means of the mirror. Instead of the axial movement of the mirror 201 it is also possible to provide an oscillation of the mirror in order to change the direction of the reflecting ray.

If the mirror for example is mounted on a fork in an oscillating way about an axis connecting the two branches of the fork, then a member extensible by heating effect can control the oscillation of the mirror if dynamically coupled to the mirror or to a radial arm of the axis of oscillation of the mirror such that the expansion action occurs in a direction perpendicular to the axis of oscillation and with at least a component perpendicular to the oscillating arm and between said arm and a stationary match. The mechanism itself that is not shown because it is simple to understand may be used for rotating a shaft supporting the mirror oriented in the axial direction of the needle or probe that is in a direction perpendicular to the axis of oscillation of the fork.

FIG. 3 shows a third variant embodiment wherein the irradiating tip is composed of shape memory tubular members 5 each one housing an optical fiber whose laser irradiation is emitted at tips 105 and/or also laterally diffused. Due to thermal effect heating the tissue adjacent to said tubular arms, when the latter change their shape, the laser irradiaton is distributed on different portions of the lesion tissue area to be treated. A main optical fiber reaching the end of the needle or probe at which two, three or more tubular members 5 are provided independent one from the other and having such an arrangement to produce a kind of umbrella, divides into or extends with an optical fiber for each one of said tubular members 5. Tubular members are shape memory ones, and such that when temperature progressively increases they radially outwardly move projecting and/or diffusing the irradiation in various portions of the area to be treated, as the temperature thereof changes. The change in temperature of tubular members may be due both to indirect heating thereof by the tissue surrounding them both to direct heating by irradiation passing through them and/or due to a combination of said effects.

FIG. 4 shows a third variant embodiment wherein the distribution of the heating effect on the size of the area comprising the tissue to be treated occurs by the fact of making the needle dynamic with an umbrella of solid or hollow tines or wires composed of shape memory material indicated by 6. Therefore it is a peripheral area of the lesion tissue, i.e of the tumor. When said area reaches a certain temperature the umbrella of tines or wires brings itself more and more towards the central area.

A variant can provides the temperature of the irradiating tip to be taken there being provided means for operating a translation returning the needle for a length equal to the diameter of the area considered to be treated. In this case, the umbrella of solid or hollow tines or wires 6 is enlarged or reduced by means of a progressive unthreading or advancing action of the needle with said wires or tines with respect to a tubular tip. Tines are hot fibers of thermally conducting material or hollow ones for the light to be passed through, and are elastically pre-loaded in the direction of a movement and/or bending in an outward radial direction with respect to the axis of the needle when the needle is retracted with respect to the end of a cannula, then tines or wires are progressively brought one near the other, while a movement of the needle with respect to the end of the cannula that is a movement leading to a greater output of tines from the end if the cannula allows their progressive movement and/or progressive bending in the radial direction and outwardly with respect to the central axis of the needle and/or cannula.

It is to be noted in this case that, the axial movement of the needle with respect to the cannula results in bringing tines or wires one near the other or in allowing the mutual moving away of tines or wires with respect to the needle bearing at its end said wires or tines. This effect can be automatically obtained depending on the temperature for example by providing a cannula with an axially sliding end bushing and which is axially sliding operated by a thermally expansible material, so the bushing axially moves with respect to tines or wires depending on temperature.

A further embodiment that can be provided as an alternative to or in combination with one or more of the preceding embodiments is based on the principle of using the change of the diffusion of the electromagnetic ray by a change in the transparency and/or diffusion index that can change depending on the temperature of a diffusing member taking different conditions diffusing the electromagnetic irradiation in different area of its size depending on the local temperature in said areas.

Irradiating tips laterally outwardly diffusing the laser irradiation are known to prior art and are described in details for example in document U.S. Pat. No. 5,370,649.

By providing such a tip in combination with a covering and/or in a material changing its characteristics diffusing the laser irradiation depending on the temperature a diversified treatment can be obtained of areas of the lesion tissue depending on the temperature.

In examples of FIGS. 5 and 6, the irradiating tip has a predetermined length and it is intended to irradiate the irradiation for its length by diffusing effect and in a substantially even way.

When a portion of an area or a segment or a portion of said elongated tip 101 reaches a certain temperature the latter can reduce the diffusion coefficient and/or can completely become opaque.

In embodiment of FIG. 6 the laterally diffusing irradiating tip 101 is made of various segments 601 each of them changing its properties diffusing the electromagnetism irradiation depending on temperature. In this case, the segmented irradiating tip that changes its diffusion properties depending on the temperature allows to have a diversified treatment depending on the temperature by the fact that when temperature conditions are such that a segment changes its characteristics since it has finished the treatment of an area associated thereto having reached the predetermined changing temperature of said diffusion properties, the laser light automatically is diffused by an adjacent segment treating a different area for example a closer area.

FIG. 7 shows an embodiment of the invention wherein means for distributing the heat generated by the electromagnetic irradiation are composed of substances intended to be distributed or diffused or that permeate in time the tissue area to be treated and which substances are heated by the laser irradiation and distribute the heat by perfusing the tissue to be treated.

In this case the fluid that is heated is composed of vapour due to the heating effect of tissues by the electromagnetic irradiation. The vapour is caused to have a certain temperature and it is moved inside the area to be treated thus making a heat storing and conveying carrier.

Different ways for moving the vapour generated by the electromagnetic energy are possible, a first embodiment is what shown in FIG. 7 among the others.

In this case means for pushing said fluid can be composed of a fluid jet there being provided on the tip of the probe or needle at least a nozzle supplying said jet or said jets. FIG. 7 shows an irradiating tip of the needle to which a nozzle 7 is associated for localizely injecting an overpressure such as for example a blow by means of which the vapour is pushed.

As an alternative to or in combination with an overpressure the nozzle 7 can exert an opposite suction action.

A further variant for moving the vapour provides a mechanical pressure wave generated by a source of acoustic waves particularly ultrasound ones. Ultrasound waves have a low frequency and triangular or sawtooth pulse arrangement. That allows to push the vapour by transferring it from one to another area to be treated in the propagation direction.

In this last embodiment that can be provided separately or in combination with further arrangements of the present invention described with reference to other embodiments, it has to be noted how the vapour that currently is considered a negative element for properly making the thermal ablation by electromagnetic irradiation, is on the contrary used to obtain an advantageous effect helping the even distribution of the heat on all the area to be treated, thus overcoming drawbacks that have been discussed above to which at present the thermal ablation by electromagnetic irradiation particularly by laser light is subjected to.

1. Device for localized thermal ablation of lesion tissues, particularly tumoral tissues or the like, which device comprises:
a probe or needle intended to be positioned with the end tip at the lesion tissue or tumoral tissue area to be removed;
which probe or needle support at least a light guide as an elongated member like a thin wire or thread, one of the ends thereof is an end emitting heating electromagnetic energy and which light guide ends at said end of the probe or needle by a tip irradiating said electromagnetic energy, particularly as a laser light and the other end thereof is connected to a source generating the electromagnetic energy;
means for controlling the activation/deactivation of the source generating the electromagnetic energy.
Characterized in that in combination it comprises means for the controlled distribution of the heating action on the lesion tissue area generated by the electromagnetic energy emitted by the irradiating tip inside a volume having a predetermined size;
The said means consisting in means for varying the distribution of the emitted electromagnetic energy on at least part of the lesion tissue area as a function of the temperature measured at one or more places of the lesion tissue area and particularly at the lesion tissue area currently under treatment.

2. Device according to claim 1, characterised in that the said means for the controlled distribution of the heating action on the lesion tissue area consist in means for distributing, projecting or pointing the electromagnetic irradiation emitted by the irradiating tip on different portions of the lesion tissue area, the said means for distributing, projecting or pointing the electromagnetic irradiation emitted by the irradiating tip being adjustable relatively to the portion of the lesion tissue area to be irradiated as a function of the temperature of one or more portions of lesioned tissue area, particularly as a function of the temperature of the portion of lesioned tissue area currently under treatment and in such a way that by reaching of a predetermined temperature at the portion of the lesioned tissue area under treatment the said means distributing, projecting or pointing the electromagnetic irradiation emitted by the irradiating tip are automatically adjusted to distribute, project or point the electromagnetic irradiation on a different portion of the lesion tissue area.

3. A device according to claim 1 or 2, characterised in that the means for the controlled distribution of the heating action on the lesion tissue area generated by the electromagnetic energy emitted by the irradiating tip inside a volume having a predetermined size consist in means for automatically varying the intensity and/or the spectrum of the emitted electromagnetic energy as a function of the temperature in the irradiated lesion tissue area.

4. Device according to one or more of the preceding claims, characterized in that said active means for distributing, projecting or pointing the electromagnetic irradiation emitted by the irradiating tip on different portions of the lesion tissue area are composed of means for diffusing or concentrating or reflecting the electromagnetic irradiation which change depending on the temperature the direction and/or impression of a ray or beam of electromagnetic irradiation and the portion of the lesion tissue area illuminated by said ray or beam or upon which said ray or beam of electromagnetic energy is incident.

5. Device according to claim 4, characterized in that said means diffusing, concentrating or reflecting the electromagnetic irradiation are supported in an orientable way by means of supports whose shape and/or size depends on the temperature or changes according to temperature and which supports are in thermal contact with the surrounding environment and/or are subjected to heat due to irradiation by the electromagnetic irradiation coming from the irradiating tip.

6. Device according to one or more of claims 1 to 5, characterized in that the irradiating tip comprises at least a reflecting member that is supported in an orientable way with respect to the direction of propagation of the beam coming from said irradiating tip and/or to the direction of the axis of the light guide and whose orientation is controlled by mechanical thermal sensitive means.

7. Device according to claim 7, characterized in that means for controlling the orientation of the reflecting means is composed of a thermal sensitive shape memory member or of a bimetallic member or a member bending by a predetermined angle or a predetermined bending depending on temperature.

8. Device according to claim 6 or 7, characterized in that it comprises an irradiating tip from which an electromagnetic beam is emitted having a first direction of propagation, the reflecting member being supported in an oscillating way at a predetermined distance from said irradiating tip in the direction of propagation of the electromagnetic beam, while said reflecting member is provided coincident with said beam and it is oriented to deviate said beam in a second direction of propagation according to a predetermined angle that can change according to temperature.

9. Device according to one or more claims 6 to 8, characterized in that the reflecting member is supported in a way movable in the direction of propagation of the electromagnetic ray and depending on temperature being mounted on supporting means changing their length according to temperature.

10. Device according to one or more claims 6 to 9, characterized in that means for supporting the thermal sensitive reflecting member are under condition of thermal contact and thermal exchange with the surrounding environment.

11. Device according to one or more of the preceding claims, characterized in that the reflecting member is supported at the end of a supporting shaft that is oriented in the direction of propagation of the electromagnetic ray and that can be transversally extendible and/or pliable and/or bendable and/or flexible according to temperature.

12. Device according to one or more of the preceding claims, characterized in that the reflecting member is symmetrical by rotation about an axis coinciding with or parallel to the axis of propagation of the electromagnetic ray or it is composed of an angular sector of such symmetrical member by rotation.

13. Device according to one or more of claims 1 to 4, characterized in that the irradiating tip is composed of a member diffusing the electromagnetic irradiation having a predetermined size and it is composed of a substance having a transparency and/or a diffusion index that changes depending on temperature and taking different conditions for diffusing the electromagnetic irradiation in different areas of its size according to the local temperature in said areas.

14. Device according to claim 13, characterized in that the diffusing member is composed of a member elongated in the direction of propagation of the electromagnetic beam from the irradiating tip or longitudinal axis of the guide propagating the electromagnetic irradiation, said elongated member being composed of a material intended to diffuse the electromagnetic irradiation through the peripheral shell wall and said material being provided with a diffusion index that changes depending on the temperature between a smallest and greatest diffusion value of the input electromagnetic irradiation.

15. Device according to claim 14, characterized in that the diffusing member is covered by a material layer having a transparency or an opacity that can change depending on temperature.

16. Device according to one or more of the preceding claims 13 to 15, characterized in that the diffusing member is composed of a plurality of various diffusing members arranged one near the other and having different functions of the dependence between diffusion index and temperature.

17. Device according to one or more of the preceding claims, characterized in that it comprises means for distributing the heat generated by the electromagnetic irradiation on distributing means.

18. Device according to claim 17, characterized in that means for distributing the heat generated by the electromagnetic irradiation upon them are solid mechanical means transmitting the heat generated by the electromagnetic irradiation emitted by the irradiating tip, which means are composed of one or more wires, bands or flaps axially projecting in the direction of propagation of the electromagnetic irradiation past the irradiating tip in order to make an umbrella and which wires, bands and/or flaps are composed of a material that can be deformed according to temperature, the irradiating tip being provided with means for oriented and/or transmitting the electromagnetic beam on said wires, bands and/or flaps and/or inside thereof such that when said wires, said bands and/or said flaps progressively get warm, they change their shape and/or move in order to be open wide one with respect to the other or they move angularly radially outward.

19. Device according to claim 17, characterized in that it is provided in combination with means injecting a heat conveying fluid, for example vapour or the like, which fluid is heated by the irradiation emitted by the supplying tip.

20. Device according to claim 19, characterized in that it comprises means injecting and/or sucking a fluid pushing a further heat storing fluid provided in the treatment area.

21. Device according to claim 20, characterized in that the heat storing fluid is composed of vapour generated in the treatment area by the heating action of the electromagnetic irradiation, while injecting/sucking means generate a pushing blow and/or pulling suction for said vapour for diffusing or moving it in the direction moving it away from or in the direction approaching it to the supplying tip respectively.

22. Device according to claim 17, characterized in that it is provided in combination with means for pushing a heat conveying fluid, for example vapour or the like, which fluid is heated by the irradiation emitted by the supplying tip and which pushing means are composed of low frequency pressure waves.

23. Device according to claim 22, characterized in that said low frequency waves are composed of low frequency ultrasound pulses with triangular or sawtooth waveform.

24. Device according to claim 22 or 23, characterized in that to the supplying tip there are associated a transducer or a transducer assembly generating ultrasound pulses directly mounted on the tip or they are provided as separated unit.

25. Device according to one or more of claims 19 to 20, characterized in that means for pushing said fluid are composed of a fluid jet there being provided on the tip of the probe or needle at least a nozzle supplying said jet or said jets.

26. Method for localized thermal ablation of lesion tissues, particularly tumoral tissues or the like, which method comprises following steps:
generating an electromagnetic irradiation having a predetermined energy and frequency;
irradiating locally and for a predetermined period of time, with said electromagnetic irradiation, a lesion tissure area or a portion thereof in order to increase the temperature of the lesion tissue of said area or portion thereof to a predetermined value;
Characterized in that in combination it comprises steps controlling in an active way the distribution of the heating action on the lesion tissue area generated by the electromagnetic energy inside a volume having a predetermined size.

27. Method according to claim 26, characterized in that the control of the distribution of the heating action generated by the electromagnetic energy on the lesion tissue area is carried out by an automatic control for distributing, projecting or pointing the electromagnetic irradiation on different portions of the lesion tissue area depending on the temperature reached by said areas.

28. Method according to claim 27, characterized in that the active control of the distribution, projection or pointing of the electromagnetic irradiation on different portions of the lesion tissue area is obtained by changing diffusion or concentration or projection or reflection parameters of the electromagnetic irradiation depending on the temperature such as the direction and/or impression of a ray or beam of electromagnetic irradiation and the portion of lesion tissue area illuminated by said ray or beam or upon which said ray or beam of electromagnetic energy is incident.

29. Method according to claim 28, characterized in that the change of the direction of the electromagnetic irradiation ray and/or of the area illuminated by diffusion, is obtained by mechanical means whose shape and/or size depends on the temperature or changes depending on the temperature and the latter thermally contact the surrounding environment and/or are subjected to heating by irradiation by the electromagnetic irradiation.

30. Method according to one or more of claims 27 to 29, characterized in that the change in the diffusion of the electromagnetic ray is obtained by a change of the transparency and/or a diffusion index that can change depending on the temperature of a diffusing member taking different diffusion conditions for the electromagnetic irradiation in different areas of its size depending on the local temperature in said areas.

31. Method according to one or more of the preceding claims 27 to 30, characterized in that the distribution of heat generated by the electromagnetic irradiation is changed by moving heat transmitting means in the lesion tissue area.

32. Method according to claim 31, characterized in that the movement of heat transmission means is made by the shape memory effect depending on the temperature of heat transmitting means generated by the electromagnetic irradiation thereon.

33. Method according to claim 31, characterized in that the distribution of heat generated by the electromagnetic irradiation is obtained by moving in the treatment area a fluid, such as vapour or the like, generated in the treatment area and heated by said electromagnetic irradiation, while said movement is obtained by pushing or pulling said fluid.

34. Method according to claim 33, characterized in that means for pushing said fluid are composed of a fluid jet or a suction source.

35. Method according to claim 33, characterized in that means for pushing said fluid are composed of the mechanical pressure wave generated by an acoustic wave source particularly ultrasound ones.

36. Method according to claim 35, characterized in that ultrasound waves have a low frequency and triangular or sawtooth pulse arrangement.

37. Method according to one or more of preceding claims 33 to 36, characterized in that the substance conveying the thermal energy is composed of the vapour generated by heating the tissue by the electromagnetic ray.
